SEQUENCE LISTING (34 sequences)

SEQ ID NO: 1 - RNA, 88 nt, Homo sapiens
ugcgcuccucucagucccugagacccuaacuugugauguuuaccguuuaaauccacggguuaggcucuugggagcugcgagucgugcu

SEQ ID NO: 2 - RNA, 89 nt, Homo sapiens
accagacuuuuccuagucccugagacccuaacuugugagguauuuuaguaacaucacaagucaggcucuugggaccuaggcggagggga

SEQ ID NO: 3 - RNA, 71 nt, Homo sapiens
gccaacccaguguucagacuaccuguucaggaggcucucaauguguacaguagucugcacauugguuaggc

SEQ ID NO: 4 - RNA, 110 nt, Homo sapiens
aggaagcuucuggagauccugcuccgucgccccaguguucagacuaccuguucaggacaaugccguuguacaguagucugcacauugguuagacugggcaagggagagca

SEQ ID NO: 5 - RNA, 110 nt, Homo sapiens
ugaacauccaggucuggggcaugaaccuggcauacaauguagauuucuguguucguuaggcaacagcuacauugucugcuggguuucaggcuaccuggaaacauguucuc

SEQ ID NO: 6 - RNA, 110 nt, Homo sapiens
gcugcuggaagguguagguacccucaauggcucaguagccaguguagauccugucuuucguaaucagcagcuacaucuggcuacugggucucugauggcaucuucuagcu

SEQ ID NO: 7 - DNA, 20 bp, Artificial sequence
  Synthetic polynucleotide
aaggtgaaggcctacaagcg

SEQ ID NO: 8 - DNA, 22 bp, Artificial sequence
  Synthetic polynucleotide
tttctgctggacaggttattcc

SEQ ID NO: 9 - DNA, 20 bp, Artificial sequence
  Synthetic polynucleotide
gtgccaagggcctgaatgag

SEQ ID NO: 10 - DNA, 19 bp, Artificial sequence
  Synthetic polynucleotide
gcaaaggctccaggtctga

SEQ ID NO: 11 - DNA, 22 bp, Artificial sequence
  Synthetic polynucleotide
gtgctatggaccctacgttagt

SEQ ID NO: 12 - DNA, 22 bp, Artificial sequence
  Synthetic polynucleotide
tcattgaagttcatgcaggcaa

SEQ ID NO: 13 - DNA, 21 bp, Artificial sequence
  Synthetic polynucleotide
acaaggtccaagcctactcca

SEQ ID NO: 14 - DNA, 20 bp, Artificial sequence
  Synthetic polynucleotide
ccgggttgttgagtgttcag

SEQ ID NO: 15 - DNA, 20 bp, Artificial sequence
  Synthetic polynucleotide
cctggaacaacccgcaatac

SEQ ID NO: 16 - DNA, 20 bp, Artificial sequence
  Synthetic polynucleotide
ttccccaacctcagtcatgc

SEQ ID NO: 17 - DNA, 20 bp, Artificial sequence
  Synthetic polynucleotide
cctctgatcgatctgccctc

SEQ ID NO: 18 - DNA, 23 bp, Artificial sequence
  Synthetic polynucleotide
tcttcagtaccggaagctgttac

SEQ ID NO: 19 - DNA, 20 bp, Artificial sequence
  Synthetic polynucleotide
ggtttgctttggctcactcg

SEQ ID NO: 20 - DNA, 20 bp, Artificial sequence
  Synthetic polynucleotide
gaacatgtcctgttgctggc

SEQ ID NO: 21 - DNA, 20 bp, Artificial sequence
  Synthetic polynucleotide
gagcccactgtggtggatga

SEQ ID NO: 22 - DNA, 21 bp, Artificial sequence
  Synthetic polynucleotide
tctttgccgatggtgtagtgg

SEQ ID NO: 23 - DNA, 20 bp, Artificial sequence
  Synthetic polynucleotide
gccatgcaggacacaatcaa

SEQ ID NO: 24 - DNA, 20 bp, Artificial sequence
  Synthetic polynucleotide
gcaatctcgatgtccaaggc

SEQ ID NO: 25 - DNA, 20 bp, Artificial sequence
  Synthetic polynucleotide
ccgctatcacctgctgttcc

SEQ ID NO: 26 - DNA, 21 bp, Artificial sequence
  Synthetic polynucleotide
acattttgctggcctgctcac

SEQ ID NO: 27 - DNA, 20 bp, Artificial sequence
  Synthetic polynucleotide
tgcttcagcggaggaagttt

SEQ ID NO: 28 - DNA, 20 bp, Artificial sequence
  Synthetic polynucleotide
aagatgcgccgcacattatc

SEQ ID NO: 29 - RNA, 23 nt, Mus musculus
uauaguuuuggaucucucagggg

SEQ ID NO: 30 - RNA, 22 nt, Homo sapiens
ucccugagacccuaacuuguga

SEQ ID NO: 31 - RNA, 23 nt, Mus musculus
caugcaguggaagaaacacugga

SEQ ID NO: 32 - RNA, 23 nt, Homo sapiens
cccaguguucagacuaccuguuc

SEQ ID NO: 33 - RNA, 23 nt, Mus musculus
aagguacuuugaaacauguagca

SEQ ID NO: 34 - RNA, 21 nt, Artificial sequence
  Synthetic polynucleotide
agcuacaucuggcuacugggu

(19) United States
(12) Patent Application Publication
(10) Pub. No.: US 2018/0072993 A1
(43) Pub. Date: Mar. 15, 2018

(54) METHODS FOR ENHANCING MATURATION OF CARDIOMYOCYTES

(71) Applicant: Academia Sinica, Taipei (TW)

(72) Inventors: Patrick C.H. Hsieh, Taipei (TW); Desy S. Lee, New Taipei City (TW)

(73) Assignee: Academia Sinica, Taipei (TW)

(21) Appl. No.: 15/699,011

(22) Filed: Sep. 8, 2017

Related U.S. Application Data
(60) Provisional application No. 62/385,298, filed on Sep. 9, 2016.

(51) Int. Cl. C12N 5/077; C12Q 1/68; A61K 35/34; G01N 33/50

(52) U.S. Cl. CPC C12N 5/0657; C12Q 1/6881; A61K 35/34; C12N 2501/50; C12N 2501/65; C12Q 2600/158; C12N 2502/28; G01N 33/5061

(57) ABSTRACT

Methods for promoting maturation of cardiomyocytes, involving introducing a microRNA combination containing miR-125b, miR-199a, miR-221, and/or miR222, or suppression of ErbB4 in immature cardiomyocytes, which may be co-cultured with endothelial cells.

### RELATED APPLICATION

This application claims the benefit of the filing date of U.S. Provisional Application No. 62/385,298, filed on Sep. 9, 2016, the content of which is herein incorporated by reference in its entirety.

### BACKGROUND OF DISCLOSURE

Cells differentiated from embryonic stem (ES) cells and induced pluripotent stem (iPS) cells can form ideal platforms for in vitro disease modeling, and for the development and testing of new therapeutics. These tools are of greatest use in fields where primary human cells and tissues are not readily available, such as in cardiac or neurological research. However, one major obstacle is that cells derived from ES/iPS cells are generally immature and tend to display the structural and functional attributes of fetal cells, rather than the adult phenotype (Blum et al., Nat. Biotechnol., 2012, 30, 261-264; Maroof et al., Cell Stem Cell, 2013, 12, 559-572; Yang et al., Circ. Res., 2014, 114, 511-523).

Cardiac disease is one of the greatest causes of morbidity and mortality in the world (Lloyd-Jones et al., Circulation, 2010, 121, 948-954). Protocols have been developed to differentiate pluripotent stem cells into cardiomyocytes (CMs); however, these differentiated cells remain immature in both morphology and performance. This is unsurprising, given that neonatal human cardiomyocytes require many years of maturation in vivo before fully adopting the adult phenotype, whereas ES/iPS-CM are differentiated in only a matter of weeks (Peters et al., Circulation, 1994, 90, 713-725). An immature pluripotent cell-derived cardiomyocyte exhibits a rounded cell shape, with disorganized contractile apparatus, a single nucleus, limited calcium handling ability, and a resting membrane potential of around ≈−60 mV. By contrast, a mature adult cardiomyocyte is significantly larger and rod-shaped, with highly organized sarcomeres, polarized Connexin-43, and a more negative resting potential of around ≈−90 mV. A larger proportion of cells can also be multinucleated (Yang et al., Circ. Res., 2014, 114, 511-523). A fully mature cardiomyocyte also shows regularly spaced transverse tubules (T-tubules), which are essential for proper excitation-contraction coupling through even distribution of action potentials (Ziman et al., J. Mol. Cell Cardiol., 2010, 48, 379-386).

Due to these structural and functional differences, cells which have been differentiated from pluripotent stem cells in vitro may be unreliable when used for disease modeling, drug screening, or cell-based therapies (Kim et al., Nature, 2013, 494, 105-110; Laflamme et al., Nat. Biotechnol., 2005, 23, 845-856; Navarrete et al., Circulation, 2013, 128, S3-S13; Yang et al., Circ. Res., 2014, 114, 511-523). This is a significant problem, given that previously undetected cardiac toxicity is one of the leading causes of drug development failure (Navarrete et al., Circulation, 2013, 128, S3-S13). It is therefore of great importance to develop new methods for producing mature cardiomyocytes.

### SUMMARY OF DISCLOSURE

The present disclosure is based, at least in part, on the unexpected finding that certain miRNAs, for example, miR-125b, miR-199a, miR-221, and miR-222 enhanced maturation of cardiomyocytes, which may be co-cultured with endothelial cells and the activity of the miRNA may be due to suppression of ErbB4.

Accordingly, one aspect of the present disclosure features a method for enhancing cardiomyocyte maturation, the method comprising: (i) transfecting into immature cardiomyocytes one or more microRNA oligonucleotides, which include miR-125b oligonucleotide (e.g., a miR-125-5p oligonucleotide), a miR-199a oligonucleotide (e.g., a miR-199a-5p oligonucleotide), a miR-221 oligonucleotide, and a miR-222 oligonucleotide; thereby producing mature cardiomyocytes. In some examples, a combination of a miR-125b-5p oligonucleotide, a miR-199a-5p oligonucleotide, a miR-221 oligonucleotide, and a miR-222 oligonucleotide can be used in the method described herein to enhance maturation of cardiomyocytes.

In another aspect, the present disclosure provides a method for enhancing cardiomyocyte maturation, the method comprising: (i) inactivating ErbB4 in immature cardiomyocytes, thereby producing mature cardiomyocytes. In some embodiments, the inactivating step can be performed by introducing into the immature cardiomyocytes an interfering RNA which targets ErbB4.

In some embodiments, the immature cardiomyocytes may be derived from embryonic stem cells, for example, human embryonic stem cells or mouse embryonic stem cells. When desired, the immature cardiomyocytes may be co-cultured with endothelial cells, which can be of the same species as the immature cardiomyocytes, or from a different species as the immature cardiomyocytes.

Any of the methods described herein may further comprise (ii) measuring the level of major histocompatibility complex (MHC) alpha chain and/or the level of MHC beta chain in the cardiomyocytes after step (i); and (iii) determining maturation of the cardiomyocytes based on the level of the MHC alpha chain and/or the level of the MHC beta chain. In some examples, the level of cardiomyocyte maturation is determined based on the ratio between the level of the MHC alpha chain and the level of the MHC beta chain.

Alternatively or in addition, any of the methods described herein may further comprise transplanting the mature cardiomyocytes to a subject in need thereof. In other examples, the method may further comprise contacting the mature cardiomyocytes with a candidate agent to determine the impact of the candidate agent on the mature cardiomyocytes.

Also described herein are mature cardiomyocytes produced by any of the methods described herein. Such mature cardiomyocytes can be used for therapeutic purposes (e.g., for treating cardiovascular diseases) or for research purposes (e.g., drug screening or efficacy testing).

The details of one of more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawing forms part of the present specification and are included to further demonstrate certain aspects of the present disclosure, which can be better understood by reference to one or more of these drawings in combination with the detailed description of specific embodiments presented herein.

FIG. 1 shows the results of mouse embryonic stem cell-derived cardiomyocytes co-cultured with endothelial cells. Panel A: Stains show mES-CMs co-cultured with ECs took on an elongated cell shape and improved their alignment, compared to when they were cultured alone. Panel B and Panel C: Both immunofluorescence and ultrastructural analyses of co-cultured mES-CMs revealed myofibrils with properly aligned Z-bands, whereas ES-CMs cultured alone contained only nascent myofibrils and poorly aligned, immature Z-bodies. Panel D: Graphs show the rising slope and time to peak were not significantly different between the two groups, however a faster τ decay indicates improved kinetics of Ca²⁺ clearance from the cytosol of ES-CMs cultured with ECs.

FIG. 2 shows the results of microRNAs delivered to mouse embryonic stem cell-derived cardiomyocytes. Panel A: The candidates chosen were most differentially upregulated in EC co-cultured ES-CMs in three independent experiments: These were miR-125b-5p, miR-199a-5p, miR-221 and miR-222. Panel B: The expression levels of these miRNAs were then confirmed using quantitative RT-PCR. Panel C: miR-combo transfected mES-CMs contained more aligned, mature myofibrils in the cytoplasm. Panel D: miR-combo transfected mES-CMs also showed more organized sarcomeric structures with the presence of I bands and (Panel E) mitochondrial inner membranes with more well-formed cristae. Panel F: An increased protein level of the cardiomyocyte gap junction component CONNEXIN-43 was also measured. (Panel G) Examination of calcium transients revealed a significant increase in the decay rate of Ca²⁺ clearance, as evidenced by a decrease in τ decay time.

FIG. 3 shows the downregulation of ErbB4 following EC co-culture and miR-combo delivery. Panel A-Panel C: Graphs and a SDS-Page gel show the results of significant reduction in the expression of ErbB4 in ES-CMs following EC co-culture and following miR-combo delivery. Panel D: A graph shows that ErbB4 expression declined during the prenatal stage of cardiac development, which inversely correlates with the expression of these four miRNAs. Panel E: A graph shows that each individual miRNA can significantly reduce luciferase activity, suggesting that each miRNA in the miR-combo can target ErbB4. Panels FI: Results show a higher α-/β-MHC ratio using the same assays to assess the maturity indices of each group, increased gene and protein expression of CONNEXIN-43 and more aligned sarcomeres following ErbB4 knockdown.

FIG. 4 shows the maturation-associated changes in human ES-CMs promoted by the miR-combo. Panels A-C: hES-CMs showed several morphological changes associated with increased maturation. hES-CMs transfected with miR-combo showed more organized sarcomeres, a larger cell area and a higher binucleation ratio compared to scramble miRNA transfected controls, although they still retained a rounded cell shape. Panels D-F: Graphs show these changes were also accompanied by a more negative resting membrane potential and a larger amplitude of action potential, as measured by whole cell patch clamp. Panels G-J: Data showed lower expression of ANF and higher expression of CONNEXIN-43 and KIR2.1 following miR-combo delivery. Panels K-L: Results show that after two months, a more organized distribution of Connexin-43 and enlarged cell size visible in miR-combo treated hES-CMs, compared to controls which were cultured for the same period of time.

FIG. 5 shows the generation of mouse ES-CMs and the co-culture system. Panels A-C: The timeline of experimental program in differentiating these mES-CMs and immunofluorescence staining showing the morphology of mES-CMs with GFP. Panel D: A graph showing the larger cell area of mES-CMs after co-culture with ECs. Panels E-F: Graphs showing higher α-/β-MHC ratio and Serca2a of mES-CMs which indicates the more mature status of these mES-CMs.

FIG. 6 shows how these miRNAs were identified from mES-CM and EC co-culture. Panel A: Heat map illustration showing all miRNAs that were >1.5× upregulated in mES-CMs co-cultured with ECs in three independent experiments. Panel B: Graphs showing the expression of each miRNA in the candidates compared to U6 following co-culture. Panel C: Graphs showing the expression level of each miRNA at E10.5, E14.5 and E17.5 in mouse heart using quantitative RT-PCR. Panels D-E: Graphs and immunofluorescence staining of mES-CMs cultured alone, in EC-conditioned medium (CondMed), with EC lysate, and on the matrix of ECs.

FIG. 7 shows the maturation analyses of mES-CM following miR-Combo transfection. Panel A: Timeline of experimental program for miRNA or siRNA transfection in mES-CMs. Panel B: Graphs show higher α-/β-MHC ratio of mES-CMs which indicates the more mature status of mES-CMs. Panels C-D: Immunofluorescence and graph showing more elongated cells and higher percentage of mature myofibrils in miR-Combo group. Panels E-F: Ultrastructural images of mES-CMs showing improved sarcomere organization and more mature mitochondria morphology in mES-CMs following miR-Combo transfection

FIG. 8 shows ErbB4 as a common target of four miRNAs. Panel A: The predicted binding site of each miRNAs (miR-125b, miR-199a, miR-221, and miR-222) in the 3′UTR of ErbB4 mRNA. From top to bottom, sequences correspond to SEQ ID NOs: 29-34. Panel B: A graph showing the knockdown efficiency of ErbB4 in mES-CMs transfected with siRNA. Panels C-D: Graphs showed the enriched GO terms for upregulated genes determined by microarray analysis with the confirmation of the microarray result.

### DETAILED DESCRIPTION OF DISCLOSURE

Endothelial cells (EC) play a role in promoting cardiomyocyte (CM) survival and organization. See, e.g., Brutsaert, Physiol. Rev., 2003, 83, 59-115; Hsieh et al., Annu. Rev. Physiol., 2006, 68, 51-66; and Hsieh et al., Circulation, 2006, 114, 637-644. Co-culture of immature cardiomyocytes with ECs was found to improve ES-CM maturation. Lee et al., Cell Reports, 12(12):1960-1967 (2015) and Hsieh et al., Circulation 120 (suppl 18), Abstract 3873 (2009).

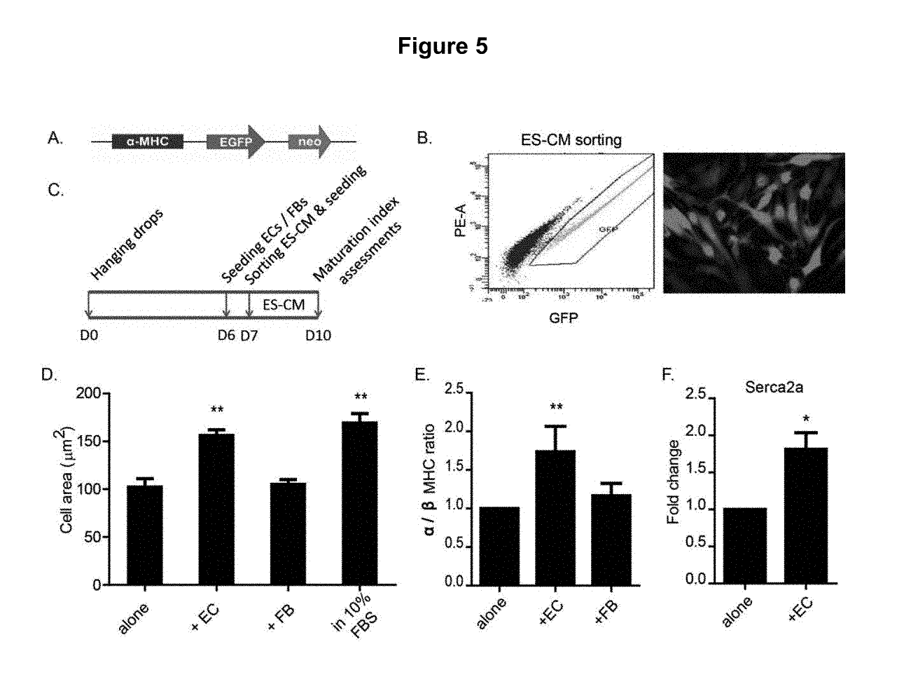

Given the heterogeneity of ECs, it was sought to determine whether different ES-CM maturation effects could be triggered by different sources of ECs upon co-culture. Surprisingly, it was found that, regardless of origin (heart, fat, or aorta) or even species (mouse, rat, pig, or human), ECs could enhance the maturity of cardiomyocytes derived from embryonic stem cells (ES-CMs) using the co-culture system. In addition, this effect could not be replicated by co-culture with fibroblasts. See, e.g., FIG. 5, Panel D to Panel E. Therefore, it was postulated that a common property of the various ECs contributes to the maturation effect of ES-CMs.

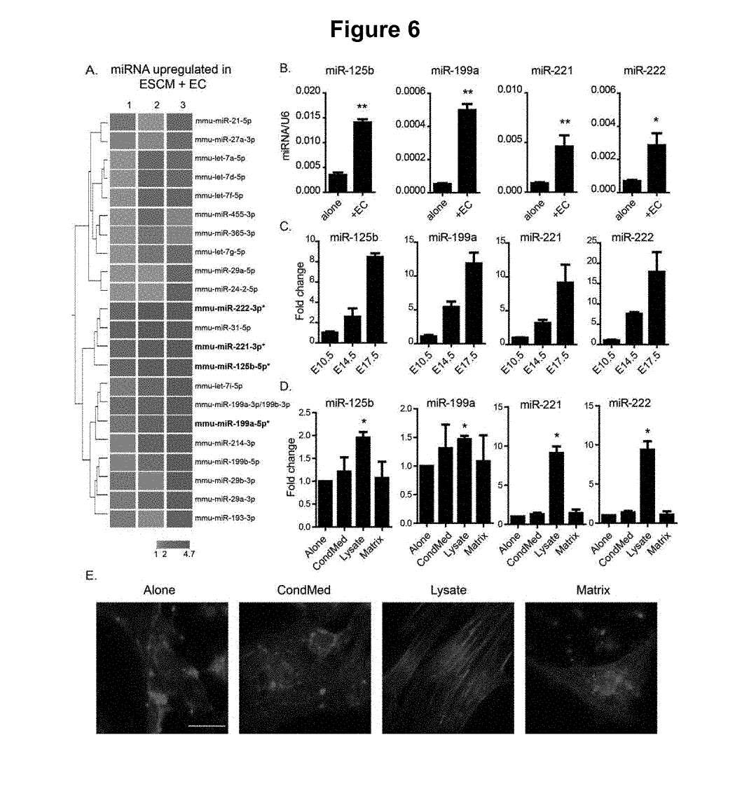

The present studies revealed that the co-culture with ECs increases the level of miR-combo miRNAs in ES-CMs cultured under a variety of conditions. ES-CM cells were cultured in a EC-conditioned medium, with a lysate of freeze-thawed ECs, or on an extracellular matrix coated with ECs. Interestingly, it was found that only the lysate of endothelial cells was able to significantly enhance expression of the four miRNAs described herein (FIG. 6, Panel D). ES-CMs co-cultured with a EC conditioned medium or on matrix alone showed only an immature phenotype, whereas ES-CMs cultured with EC lysate showed much more organized sarcomeric structures (FIG. 6, Panel E). This indicates that the main maturation-enhancing effect is not derived through a secreted factor or via any matrix-driven effect. It is known that miRNAs can be transferred directly between cells through gap junctions (Katakowski et al., Cancer Res., 2010, 70, 8259-8263), and it is suggested that this may be the mechanism by which EC co-culture affects ES-CM miRNA concentrations.

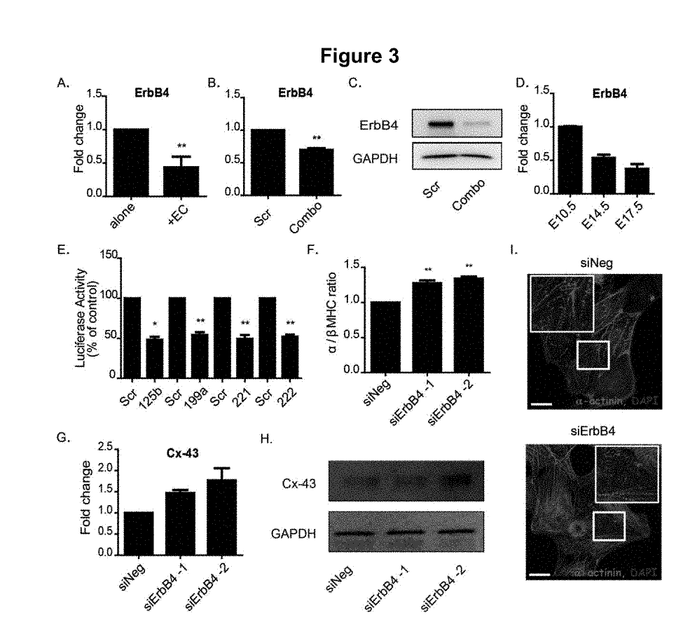

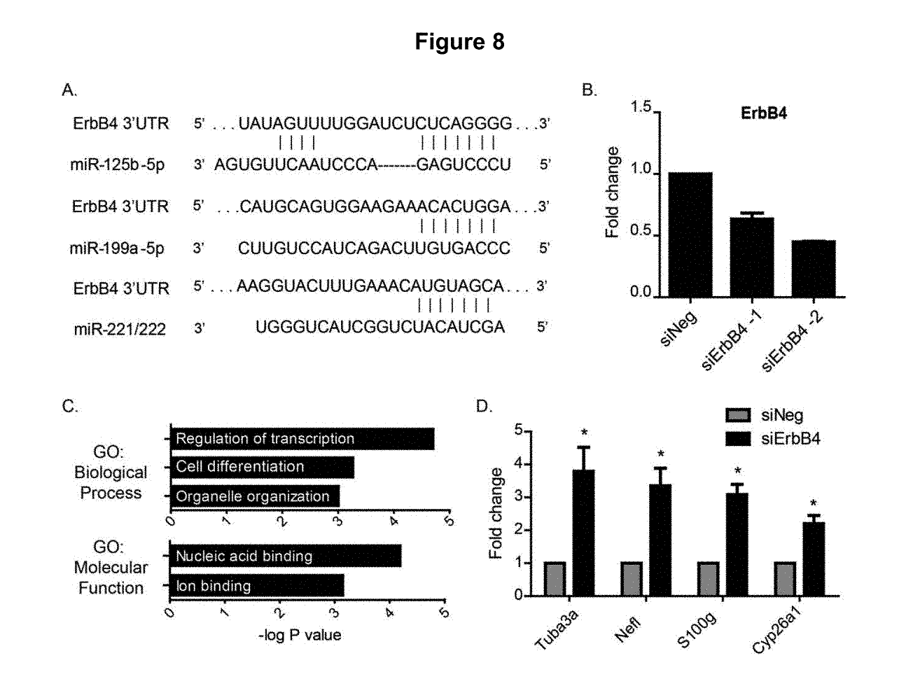

Without being bound by theory, all four of the miRNAs described herein may independently repress ErbB4 and that expression of these four miRNAs increases during cardiac development (FIG. 6, Panel C and FIG. 3, Panel D). In order to provide a degree of insight into the impact of ErbB4 knockdown, a microarray analysis was carried out following siErbB42 delivery to ES-CMs. GO analysis revealed regulation of transcription, cell differentiation, organelle organization, and ion binding were upregulated. Several potentially relevant genes were then chosen (Tuba3a, Nefl, S100g, and Cyp26a1) and their upregulation was confirmed by quantitative RT-PCR (FIG. 8, Panel C to Panel D).

These findings provide a simple and rapid method for enhancing the maturity of cardiomyocytes, such as those derived from murine and human pluripotent cells, -via co-expressing the combination of microRNAs miR-125b, miR-199a, miR-221, and miR-222, or suppressing ErbB4. Although the adult phenotype (i.e. rod-shaped cells with a resting potential −90 mV, peak action potential of 300 mV, and T-tubules) was not fully recapitulated, delivery of the miR-combo yielded a population of cells which displayed many more aspects of maturation than cells cultured under standard conditions.

The methods described herein have a number of advantages. Firstly, it is based on real physiological mechanisms, which are supported by the interactions occurring between cardiac endothelial cells and cardiomyocytes. Secondly, miRNAs have the potential to regulate many targets and complex pathways and are still more selective than many small molecule agents. Thirdly, miRNAs are easy to manipulate ex vivo and thus are suitable agents for increasing in vitro maturation of ES-CMs. Finally, these effects were demonstrated to persist for at least two months after a single miR-combo delivery.

Accordingly, one aspect of the present disclosure relates to methods for promoting cardiomyocyte maturation, which may involve either expressing of one or more of the microRNAs described herein (a miR-125b-5p oligonucleotide, a miR-199a-5p oligonucleotide, a miR-121 oligonucleotide, and a miR122 oligonucleotide), or suppressing ErbB4 in immature cardiomyocytes. In some embodiments, a combination of all four miRNA oligonucleotides described herein can be used for promoting cardiomyocyte maturation.

The immature cardiomyocytes for use in the methods described herein can be obtained from a suitable species (e.g., human, mouse, rat, pig, etc.) via routine methods. For example, such cardiomyocytes can be differentiated from cardiomyocyte precursor cells by culturing the precursor cells in a suitable culture medium under suitable conditions. The culture medium may comprise one or more growth factors to induce differentiation of the precursor cells to form immature cardiomyocytes. Alternatively, the immature cardiomyocytes may be derived from embryonic stem cells (ES cells) and/or pluripotent stem cells (iPS cells) following methods known in the art. See, e.g., Kim et al., Nature, 494:105-110 (2013); Laflamme et al., Nat. Biotechnol. 23:845-856 (2005); Navarrete et al., Circulation 128:S3-S13 (2013); and Yang et al., Cir. Res. 114:511-523 (2014). See also Example 1 below. In some instances, cardiac differentiation can be induced by the hanging drop technology using ascorbic acid. See, e.g., Hsieh et al., Rev. Physiol. 68:51-66 (2006).

In some embodiments, the immature cardiomyocyte cells can be in contact with one or more of four microRNA oligonucleotides, a miR-125b oligonucleotide (e.g., a miR-125b-5p oligonucleotide)-5p, a miR-199a oligonucleotide (e.g., a miR-199a-5p oligonucleotide), a miR-221 oligonucleotide, and a miR-222 oligonucleotide to promote maturation.

MicroRNAs (miRNAs) are a group of small noncoding RNAs that post-transcriptionally suppress the expression of their target genes in a sequence-specific manner. This type of RNAs has profound effects in orchestrating cardiac development, function, and pathological responses to injury (Cordes et al., Circ. Res., 2009, 104, 724-732; Small et al., Circulation, 2010, 121, 1022-1032).

Naturally occurring miR-125b e.g., miR-125b-5p, miR-199a (e.g., miR-199a-5p), miR-221, and miR-222 are well-characterized microRNAs in the art. Information for these microRNAs can be obtained from the microRNA database (www.mirbase.org) or from GenBank. The nucleotide sequences of human miR-125b-5p, miR-199a-59, miR-221, and miR-222 (precursors) are provided below. The boldfaced/underlined sequences represent the mature miRNAs. Naturally occurring miR-125b-5p has two putative precursors, miR-125b-1 (SEQ ID NO: 1) and miR-125b-2 (SEQ ID NO: 2). Similarly, miR-199a-5p also has two putative precursors, miR-199a-1 (SEQ ID NO: 3) and miR-199a-2 (SEQ ID NO: 4).

Human microRNA 125b (hsa-miR-125b-1 and
hsa-miR-125b-2)
(hsa-miR-125b-1; SEQ ID NO: 1)
UGCGCUCCUCUCAGUCCCUGAGACCCUAACUUGUGAUGUUUACCGUUUA
AAUCCACGGGUUAGGCUCUUGGGAGCUGCGAGUCGUGCU
(has-miR-125b-2, SEQ ID NO: 2)
ACCAGACUUUUCCUAGUCCCUGAGACCCUAACUUGUGAGGUAUUUUAGU
AACAUCACAAGUCAGGCUCUUGGGACCUAGGCGGAGGGGA
Human microRNA 199a
(hsa-miR-199a-1; SEQ ID NO: 3)
GCCAACCCAGUGUUCAGACUACCUGUUCAGGAGGCUCUCAAUGUGUACA
GUAGUCUGCACAUUGGUUAGGC
(hsa-miR-199a-2; SEQ ID NO: 4)
AGGAAGCUUCUGGAGAUCCUGCUCCGUCGCCCCAGUGUUCAGACUACCU
GUUCAGGACAAUGCCGUUGUACAGUAGUCUGCACAUUGGUUAGACUGGG
CAAGGGAGAGCA
Human microRNA221 (hsa-miR-221)
(SEQ ID NO: 5)
UGAACAUCCAGGUCUGGGGCAUGAACCUGGCAUACAAUGUAGAUUUCUG
UGUUCGUUAGGCAACAGCUACAUUGUCUGCUGGGUUUCAGGCUACCUGG
AAACAUGUUCUC
Human microRNA 221 (hsa-miR-222)
(SEQ ID NO: 6)
GCUGCUGGAAGGUGUAGGUACCCUCAAUGGCUCAGUAGCCAGUGUAGAUC
CUGUCUUUCGUAAUCAGCAGCUACAUCUGGCUACUGGGUCUCUGAUGGCA
UCUUCUAGCU

The terms “miR-125b oligonucleotide,” “miR-125b-5p oligonucleotide,” “miR-199a oligonucleotide,” “miR-199a-5p oligonucleotide,” “miR-221 oligonucleotide,” and “miR-222 oligonucleotide” as used herein refer to oligonucleotide molecules mimicking the function of naturally occurring miR-125b-5p, miR-199a-5p, miR-221, and miR-222, respectively, i.e., suppressing the expression of target genes of these microRNAs. Such oligonucleotide molecules comprise an nucleotide sequence (e.g., 21-24 nt in length) that is substantially similar to the nucleotide sequence of the corresponding mature microRNA as identified above such that the oligonucleotide molecule, when delivered into a target cell, could hybridize with the target mRNA, thereby leading to RNA silencing and thus suppressing the expression of the target gene.

“Substantially similar” means that the sequence comprised by a miRNA oligonucleotide shares a high level of homology to the sequence of the corresponding naturally-occurring miRNA (mature form) such that, like the mature naturally-occurring miRNA, the miRNA oligonucleotide described herein can form duplex with the target mRNA and thus suppress expression of the target mRNA/gene. In some instances, the miR-125b (e.g., miR-125b-5p), miR-199a (e.g., miR-199a-5p), miR-221, and miR-222 oligonucleotides described herein may comprise a nucleotide sequence at least 80% (e.g., at least 85%, 90%, 95%, or above) identical to the corresponding nucleotide sequence of mature miR-125b, miR-199a, miR-221, and miR-222 noted above. The “percent identity” of two nucleic acids is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST® nucleotide searches can be performed with the NBLAST program, score=100, wordlength-12 to obtain nucleotide sequences homologous to the nucleic acid molecules of the invention. Where gaps exist between two sequences, Gapped BLAST® can be utilized as described in Altschul et al., Nucleic Acids Res. 25(17):3389-3402, 1997. When utilizing BLAST® and Gapped BLAST® programs, the default parameters of the respective programs (e.g., XBLAST and NBLAST) can be used.

In some instances, miR-125b (e.g., miR-125b-5p), miR-199a (e.g., miR-199a-5p), miR-221, and miR-222 oligonucleotides is similar to the mature miR-125b-5p, miR-199a-5p, miR-221, and miR-222 sequences. In some examples, the miRNA oligonucleotide described herein consists of a sequence that has the same length as the corresponding mature naturally occurring miRNA. In some examples, the miRNA oligonucleotide described herein has a slightly longer length as the mature naturally occurring miRNA, for example having less than 3 nucleotides at either the 5′ end, 3′ end, or both. Alternatively or in addition, the miRNA oligonucleotide described herein, either has the same length as or slightly longer than the mature naturally occurring miRNA, shares a high sequence identity (e.g., at least 80%, 85%, 90%, 95%, or above) as the mature naturally occurring miRNA.

In other instances, the miRNA oligonucleotide described herein is a precursor oligonucleotide, which can produce a mature miRNA oligonucleotide capable of suppressing the expression of a target mRNA/gene via the miRNA processing machinery either in vivo (e.g., inside cells) or in vitro. Such precursor oligonucleotides may comprise a core sequence that is identical or substantially similar to the sequence of the mature naturally occurring microRNA and form a hairpin structure, in which the sequence corresponding to the mature microRNA is located at the stem region. When delivered into cells, such precursor microRNAs can be processed to generate a mature microRNA, which can bind to a target mRNA to suppress target gene expression. In some examples, a precursor oligonucleotide for use in the method described herein may comprise a core sequence identical or substantially similar to the mature sequence of one of miR-125b, miR-199a, miR-221, and miR-222 and share a total sequence identity of at least 80% (e.g., 85%, 90%, 95%, or above) to the reference microRNA (in precursor form).

In other embodiments, promoting cardiomyocyte maturation can be achieved by suppressing Receptor tyrosine-protein kinase erb-4 (ErbB4) in immature cardiomyocytes as described herein. ErbB4 is a receptor tyrosine kinase belonging to the epidermal growth factor receptor subfamily. It is a single-pass type I transmembrane protein with multiple furin-like cysteine rich domains, a tyrosine kinase domain, a phosphotidylinositol-3 kinase binding site and a PDZ domain binding motif. In humans, this receptor is encoded by the ErbB4 gene. Human ErbB4 protein and the coding gene are described in GenBank accession numbers AAI43750 and mRNA L07868, the relevant disclosures of each of which are incorporated by reference herein.

In some embodiments, suppressing ErbB4 can be achieved by inhibiting the activity of the ErbB4 receptor activity. For example, the immature cardiomyocytes can be treated with an ErbB4 antagonist, such as an anti-ErbB4 antibody (e.g., a full-length antibody or an antigen binding fragment thereof) or a small molecule that inhibits the activity of ErbB4. Examples of ErbB4 inhibitors include, but are not limited to, AST-1306, poziotinib, AEE788, AC480, TAK-285, and lapatinib.

In some embodiments, suppressing ErbB4 can be achieved by inhibiting the expression of ErbB4, for example, via RNA interference using an interfering RNA that targets the ErbB4 messenger RNA. RNA interference (RNAi) is a process in which a dsRNA directs homologous sequence-specific degradation of messenger RNA. In mammalian cells, RNAi can be triggered by 21-nucleotide duplexes of small interfering RNA (siRNA) without activating the host interferon response. An interfering RNA that targets ErbB4 can be a siRNA (containing two separate and complementary RNA chains) or a short hairpin RNA (an RNA chain forming a tight hairpin structure), both of which can be designed based on the sequence of the target ErbB4 gene, which is well known in the art, by routine technology. See, e.g., Krutzfeldt et al., Nature 438 (7068): 685-689, 2005; and Czech, New England Journal of Medicine 354(11):2, 2006.

ErbB4 suppression may be measured using conventional methods. For example, ErbB4 expression may be determined before and after a perturbation. Western blot analysis with an anti-ErbB4 antibody may be used to determine protein expression. Alternatively or in addition, quantitative polymerase chain reaction analysis may be used to determine ErbB4 RNA expression. The expression of genes, such as Tuba3a, Nefl, S100g and Cyp26a1 may also be evaluated to determine the level of ErbB4 suppression.

Any of the microRNA oligonucleotides and/or interfering RNA molecules targeting ErbB4 as described herein can be prepared via conventional methods, e.g., chemical synthesis or in vitro transcription. When necessary, the oligonucleotide described herein can include non-naturally-occurring nucleobases, sugars, or covalent internucleoside linkages (backbones). Such a modified oligonucleotide confers desirable properties such as enhanced cellular uptake, improved affinity to the target nucleic acid, and increased in vivo stability.

In one example, the oligonucleotide to be used in a method as described herein has a modified backbone, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In another example, the microRNA oligonucleotides and/or the interfering RNA targeting ErbB4 described herein can include one or more substituted sugar moieties. Such substituted sugar moieties can include one of the following groups at their 2′ position: OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C₁ to C₁₀ alkyl or C₂ to C₁₀ alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

In yet another example, the microRNA oligonucleotides and/or the interfering RNA targeting ErbB4 described herein may include one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y. S., Chapter 15, Antisense Research and Applications, pages 289-302, CRC Press, 1993. Certain of these nucleobases are particularly useful for increasing the binding affinity of the oligonucleotide to miR-138. These include 5-substituted pyrimidines, 6-azapyrimidines and N-2, N-6 and O-6 substituted purines (e.g., 2-aminopropyl-adenine, 5-propynyluracil and 5-propynylcytosine). See Sanghvi, et al., eds., Antisense Research and Applications, CRC Press, Boca Raton, 1993, pp. 276-278). Alternatively or in addition, the microRNA oligonucleotides and/or the interfering RNA targeting ErbB4 described herein may include one or more locked nucleotides (LNAs).

Any of the microRNA oligonucleotides and/or the interfering RNA molecules targeting ErbB4 can be introduced into cardiomyocytes via conventional methods, for example, a routine transfection approach. Alternatively, a vector designed for producing such an oligonucleotide can be delivered into cardiomyocytes, in which the microRNAs and/or the interfering RNA targeting ErbB4 can be expressed. In some instances, the functional microRNA and/or interfering RNA is delivered into the target cells to exert the intended function. Alternatively, a precursor nucleic acid can be introduced into cardiomyocytes, in which the precursor nucleic acid converts to a functional (mature) molecule.

In any of the methods described herein, the cardiomyocytes may be co-cultured with endothelial cells, which may be obtained from a suitable donor by a routine practice. The endothelial cells may be of the same origin as the cardiomyocytes, for example, both derived from humans. Alternatively, the endothelial cells and the co-cultured cardiomyocytes may be derived from different species. For example, the cardiomyocytes may be of human origin and the endothelial cells may be of a non-human origin (e.g., mouse, rat, rabbit, or a non-human primate).

In some embodiments, the method described herein may further comprise evaluating the maturation level of cardiomyocytes produced thereby. The level of cardiomyocyte maturation may be measured by conventional methods in the field, using one or more makers indicative of cardiomyocyte maturation. In one example, evaluating the level of cardiomyocyte maturation can be performed by measuring the expression levels of the alpha chain of a major histocompatibility complex (MHC), the beta chain of the MHC, or both, in the cardiomyocytes produced by the method described herein. The beta chain of an MHC complex may be used as a maker indicative of the maturation level of human cardiomyocytes. Alternatively, the alpha chain of an MHC complex may be used as a maker indicative of the maturation level of mouse cardiomyocytes.

In some examples, both the alpha chain and the beta chain of an MHC complex can be measured, one being an indicative maker and the other being an internal control. For example, the level of cardiomyocytes may be determined based on the ratio between the level of the alpha chain and the level of the beta chain, for example, α/β (e.g., for mouse cardiomyocytes) or β/α (e.g., for human cardiomyocytes). The level of the alpha chain and/or beta chain of a MHC complex in cardiomyocytes may be measured via a conventional method, for example, an immunoassay using antibodies specific to the alpha and beta chains or an RT-PCR for measuring the mRNA levels of the alpha and beta chains. An α-/β-MHC ratio or an β-/α-ratio can be calculated, which can be used as an indicator for cardiomyocyte maturation. The level of the alpha chain and/or the level of the beta chain, or a ratio thereof, may be compared with one or more control values representing the level(s) of the alpha chain and/or beta chain, or a ratio thereof in mature cardiomyocytes (e.g., human or mouse) and/or immature cardiomyocytes (e.g., human or mouse). The maturation level of cardiomyocytes produced by any of the methods described herein can thus be determined.

In some examples, the level(s) of a marker indicative of cardiomyocyte maturity such as MHC alpha and/or beta chain may be measured before and after the treatment of the miRNA oligonucleotides or the ErbB4 suppressor, or during the course of the treatment. An increase of the maker after the treatment or during the course of the treatment indicates maturation of cardiomyocytes.

Additional methods for evaluating cardiomyocyte maturation are exemplified in Examples 1 and 2 including use of a cardiac-specific alpha MHC-driven EGFP reporter, ultrastructural image analysis, and examination of calcium transients. Evaluation of gene expression for genes known to be differentially expressed in immature and adult cardiomyocytes can also be used to determine the extent of cardiomyocyte maturation. Expression of specific genes may be measured using conventional methods, such as western blot analysis (for protein expression) and quantitative polymerase chain reaction (for RNA expression). As further described in Examples 1 and 2 below, examples of genes known to be differentially expressed between immature and mature cardiomyocytes include Connexin-43, ANF, KIR2.1, and Serca2a.

The mature cardiomyocytes prepared by any of the methods described herein, which is also within the scope of the present disclosure, can be used for various purposes, for example, for therapeutic uses (e.g., treatment of cardiovascular diseases), for drug development (e.g., identifying drug candidates for use in treating cardiovascular diseases), or for research uses (e.g., explore mechanisms of cardiomyocyte differentiation and/or maturation).

The mature cardiomyocytes may be transplanted to a patient in need of the treatment for cardiac repair or for improving heart function. In this case, an effective amount of mature cardiomyocytes obtained from any of the methods described herein may be administered to a human patient suffering from a heart disease, e.g., cardiac injury, via a suitable route.

A composition comprising mature cardiomyocytes produced by the methods described herein can be delivered to a subject in need of the treatment by a convenient manner, including, but not limited to, injection, transfusion, implantation or transplantation. In some examples, the compositions described herein may be administered to a patient by intravenous (i.v.) injection. In some examples, the mature cardiomyocytes as described herein may be packed in a delivery particle. In some examples, the mature cardiomyocytes may be administered via arterial routes (e.g., internal carotid artery). The mature cardiomyocytes may be injected intramyocardially to replenish cardiomyocytes in patients with a myocardial infarction.

The optimal dosage and treatment regime for a particular patient can readily be determined by one skilled in the art of medicine by monitoring the patient for signs of disease and adjusting the treatment accordingly.

Alternatively, the mature cardiomyocytes can also be used for screening drug candidates useful in treating heart diseases and cardiac repair. In this case, the mature cardiomyocytes can be cultured in a suitable medium in the presence of a candidate agent and the impact of the candidate agent on the cardiomyocytes can be monitored. Agents that exhibit beneficial effects on cardiomyocytes, for example, prompting cardiomyocyte growth, can be identified as drug candidates. Cell proliferation may be measured with cell cycle analysis using a DNA stain, such as propidium iodide, followed by flow cytometry. Cell viability may be measured with assays such as CellTiter-Glo®. Mature cardiomyocytes may also be used to identify agents that prevent cardiac hypertrophy. For example, the mature cardiomyocytes may be used to identify agents that counteract Endothelin 1 (ET-1)-induced hypertrophy. ET-1 treatment normally results in reactivation of fetal genes in cardiomyocytes and expression of such genes as B-type natriuretic peptide (BNP). Agents that could counteract ET-1-induced hypertrophy (e.g. prevent reactivation of fetal genes) in mature cardiomyocytes would be beneficial in treating heart disease.

Further, the mature cardiomyocytes can be used as research tools for studying cardiomyocyte functions and mechanism of action of certain drugs. The information obtained from such studies would benefit the development of new therapeutic agents and therapies.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### Defined MicroRNAs Induce Aspects of Maturation in Mouse and Human Embryonic Stem Cell-Derived Cardiomyocytes

Pluripotent cell-derived cardiomyocytes have great potential for use in research and medicine, but limitations in their maturity currently constrain their usefulness. Reported here is a method for improving features of maturation in both murine and human embryonic stem cell-derived cardiomyocytes (m/hES-CMs). It has been found that co-culturing m/hES-CMs with endothelial cells improves their maturity and upregulates several microRNAs. Delivering four of these microRNAs, miR-125b-5p, miR-199a-5p, miR-221, and miR-222 (miR-combo) to m/hES-CMs resulted in improved sarcomere alignment and calcium handling, a more negative resting membrane potential, and increased expression of genes associated with mature cardiomyocytes. Although this could not fully phenocopy all adult cardiomyocyte characteristics, these effects persisted for two months after a single delivery of miR-combo.

### Experimental Procedures

(i) Routine Culture and Cardiac Differentiation of Mouse ESCs

A cardiac-specific α-MHC-driven EGFP reporter was stably transfected in mES cells using a retroviral vector (Takahashi et al., Circulation, 2003, 107, 1912-1916). Cardiac differentiation was induced using the hanging drop technique with ascorbic acid (Sigma, 100 μmol/L). On days 7-10, EGFP-positive ES-CMs were isolated and sorted by FACS (BD FACSAria). For routine culture, rat arterial endothelial cells were cultured in M199 with 15% FBS. For routine culture, mouse neonatal cardiac fibroblasts were cultured in DMEM-HG with 10% FBS. During co-culture experiments, ECs/FBs were cultured with ES-CMS in DMEM-LG with 2% FBS. ES-CMs were cultured 1:1 with ECs or cardiac fibroblasts pre-plated one day earlier (Hsieh et al., Circulation, 2006, 114, 637-644).
(ii) Exiqon miRNA Microarray

For each array, 1 vg of total RNA was labeled with Hy3 or Hy5 using the miRCURY LNA microRNA Array Labeling Kit (Exiqon). Fluorescently labeled RNA was hybridized to Exiqon miRCURY LNA miRNA arrays, v. 11.0—human, mouse & rat (Exiqon) using MAUI DNA microarray hybridization station and SL mixer (BioMicro Systems) for 16 hours at 56° C. Arrays were washed and scanned on an Axon GenePix 4000B scanner (Molecular Devices, Inc.) and GenePix results files (GPR) containing fluorescence intensities were generated using GenePix Pro 6.0 (Molecular Devices, Inc.) software. GPR files were loaded into GeneSpring 11.5 (Agilent Technologies) and normalized using the global Lowess algorithm. Flagged spots were removed from subsequent analysis. These microarray data are deposited at GEO, under accession numbers GSE69927.
(iii) Transient Transfection

ES-CMs at 60-70% confluence were transfected with pre-miR-125b, pre-miR-199a, pre-miR-221, pre-miR-222 (PM10148, PM10893, PM10337, and PM11376, respectively) or a negative control (scramble; 25 nM) using Pepmute transfection agent. For ErbB4 inhibition, siErbB4-1 and siErbB4-2 (s201303, ss201304; Ambion) were compared to a negative control using same transfection agent (25 nM).
(iv) Cardiac Differentiation and Culture of Human ESCs

Protocols were approved by the Academia Sinica Institutional Review Board. H9 cells were routinely maintained and expanded in feeder-free conditions on Matrigel-coated plates using MEF-conditioned medium with 5 ng/ml bFGF. Prior to cardiac differentiation, cells were plated at a density of 150,000 cells/cm². To induce cardiac differentiation, media was replaced with RPMI-B27-insulin and 100 ng/mL recombinant human Activin A. After 24 h, medium was changed to RPMI-B27-insulin with BMP-4 and Wnt agonist CHIR99021. Two days later, the medium was switched to Wnt antagonist Xav 939 (Yang et al., J. Mol. Cell Cardiol., 2014, 72, 296-304). Initial beating was typically observed approximately 10 days post induction. Only cell preparations containing >80% cardiac troponin T-positive cardiomyocytes were used for further experiments.
(v) Data Analysis

Data are expressed as mean±SEM. Statistical significance was determined using a 2-tailed Student's t test or ANOVA, as appropriate. Differences between groups were considered statistically significant at P values of less than 0.05.
(vi) Real-time Quantitative Polymerase Chain Reaction

Provided below are sequence of the primer sets for real-time quantitative polymerase chain reaction analysis in this study.

αMHC:

| | |
|---|---|
| 5′-AAGGTGAAGGCCTACAAGCG-3′ | (SEQ ID NO: 7) |
| and | |
| 5′-TTTCTGCTGGACAGGTTATTCC-3′ | (SEQ ID NO: 8) |
| βMHC: | |
| 5′-GTGCCAAGGGCCTGAATGAG-3′ | (SEQ ID NO: 9) |
| and | |
| 5′-GCAAAGGCTCCAGGTCTGA-3′ | (SEQ ID NO: 10) |
| ErbB4 | |
| 5′-GTGCTATGGACCCTACGTTAGT-3′ | (SEQ ID NO: 11) |
| and | |
| 5′-TCATTGAAGTTCATGCAGGCAA-3′ | (SEQ ID NO: 12) |
| Connexin-43 | |
| 5′-ACAAGGTCCAAGCCTACTCCA-3′ | (SEQ ID NO: 13) |
| and | |
| 5′-CCGGGTTGTTGAGTGTTCAG-3′ | (SEQ ID NO: 14) |
| Serca2a | |
| 5′-CCTGGAACAACCCGCAATAC-3′ | (SEQ ID NO: 15) |
| and | |
| 5′-TTCCCCAACCTCAGTCATGC-3′ | (SEQ ID NO: 16) |
| ANF: | |
| 5′-CCTCTGATCGATCTGCCCTC-3′ | (SEQ ID NO: 17) |
| and | |
| 5′-TCTTCAGTACCGGAAGCTGTTAC-3′ | (SEQ ID NO: 18) |
| KIR2.1: | |
| 5′-GGTTTGCTTTGGCTCACTCG-3′ | (SEQ ID NO: 19) |
| and | |
| 5′-GAACATGTCCTGTTGCTGGC-3′ | (SEQ ID NO: 20) |
| Tuba3a: | |
| 5′-GAGCCCACTGTGGTGGATGA-3′ | (SEQ ID NO: 21) |
| and | |
| 5′-TCTTTGCCGATGGTGTAGTGG-3′ | (SEQ ID NO: 22) |
| Nefl: | |
| 5′-GCCATGCAGGACACAATCAA-3′ | (SEQ ID NO: 23) |
| and | |
| 5′-GCAATCTCGATGTCCAAGGC-3′ | (SEQ ID NO: 24) |
| S100g: | |
| 5′-CCGCTATCACCTGCTGTTCC-3′ | (SEQ ID NO: 25) |
| and | |
| 5′-ACATTTTGCTGGCCTGCTCAC-3′ | (SEQ ID NO: 26) |
| Cyp26a1: | |
| 5′-TGCTTCAGCGGAGGAAGTTT-3′ | (SEQ ID NO: 27) |
| and | |
| 5′-AAGATGCGCCGCACATTATC-3′ | (SEQ ID NO: 28) |

(vii) Preparation of Conditioned Medium, Endothelial Cell Lysate and Extracellular Matrix.

Endothelial lysate was collected by three rounds of freeze thaw, followed by cell scraping and filtration. EC-secreted extracellular matrix was prepared by freeze-thaw lysis of cells followed by careful washing of the culture dish surface.
(viii) Dual-Luciferase Reporter Assay.

The 3′UTR segments of mouse ErbB4 containing target sites for each miRNAs were inserted into the downstream of luc2 reporter gene of pmirGLO vector (Promega). The luciferase reporters together with miRNA precursors were transfected into HEK293 cells by Pepmute (SignaGen). Cell lysates were harvested 48 hours after transfection. Firefly and renilla luciferase activities were assessed using Dual-Luciferase Reporter Assay System (Promega).
(ix) Real-Time Quantitative Polymerase Chain Reaction

RNA was extracted from ES-CMs using Trizol (Invitrogen) and reverse-transcribed using SuperScript III Reverse Transcriptase (Invitrogen). Real-time PCR was performed using a LightCycler LC480 (Roche Diagnostics) for relative quantification of αMHC and βMHC. For miRNA study, isolated RNAs were reversed-transcribed using the Taqman primer sets for miRNAs (Ambion) with MultiScribe Reverse Transcriptase (Applied Biosystems). Real-time PCR was conducted with the Taqman 2× Universal Master Mix (Applied Biosystems).
(x) Western Blotting

Total protein was collected from cells using lysis buffer. The antibodies used were anti-CONNEXIN-43 (Abcam, 1:2000), anti KIR2.1 (SantaCruz, 1:200), anti ERBB4 (SantaCruz, 1:100) and anti-GAPDH (Millipore, 1:5000).
(xi) Immunostaining and Fluorescence Microscopy

Immunostaining was performed using α-sarcomeric-actinin (1:400, Sigma) and anti-mouse-Alexa Fluor 568 (1:800, Invitrogen) antibodies. For Connexin-43 staining, anti Connexin-43 (1:500, Abcam) antibody was used. DAPI was used to counterstain nuclei. Stained cells were visualized using ImageXpress Micro Imaging XL System.
(xii) Transmission Electron Microscopy.

Cells were seeded on Aclar Embedding Film at >70% confluence. Cells were rinsed with cold 0.1 M cacodylate buffer, pH 7.2/4% sucrose/0.05% CaCl₂ (wash buffer) then fixed with 2.5% glutaraldehyde/0.1 M cacodylate buffer for 1 h and washed 3 times with wash buffer. Post fixation was conducted with 1% OsO₄ in 0.1 M cacodylate buffer. Cells were stained with 1% uranyl acetate at 4° C. for 1 h, washed, dehydrated in graded ethanols and polymerized in the Spun embedding medium for 20 h at 70° C. Imaging was performed using a Tecnai G2 Spirit TWIN transmission electron microscope.
(xiii) Measurement of Ca²⁺ Transients.

The ES-CMs were washed 3 times with Tyrode solution (150 mM NaCl, 5 mM KCl, 2.2 mM CaCl₂, 1 mM MgCl₂, 10 mM HEPES, 5 mM glucose, pH 7.3) to remove culture medium before loading with 2 μM fura-2 AM (Invitrogen) for 1 hour at room temperature in the dark. Cells were continuously perfused with Tyrode solution. The calcium ion imaging was taken by Lambda DG4. The 340- and 380-nm fluorescence signal was recorded by NIS-Elements AR Microscope Imaging software. After subtraction of background fluorescence, the data were analyzed with Origin 9 software.
(xiv) Action Potential Measurements.

Action potentials cells were recorded from using the whole-cell configuration of the patch-clamp technique with an Axopatch 200B amplifier (Molecular Devices, Sunnyvale, Calif., USA). A coverslip with the adhered cells was placed in the recording chamber and perfused with extracellular solution consisting of (in mmol/L): 143 NaCl, 5 KCl, 1 CaCl₂, 2 MgCl₂, 5 glucose, 5 HEPES (pH 7.4 with NaOH). Patch pipettes were pulled from borosilicate glass capillaries (World Precision Instruments, FL, USA) and had resistances of 1.5-3 MΩ when filled with a solution consisting of (in mmol/L): 137 KC1, 1 MgCl₂, 5 MgATP, 5 EGTA, 5 HEPES 2 Creatine 5 Phospho-creatine (pH 7.2 with KOH). The liquid junction potential was −15.5 mV.
(xv) siErbB4 Microarray.

50 ng of total RNA was amplified by a Low Input Quick-Amp Labeling kit (Agilent Technologies, USA) and labeled with Cy3 (CyDye, Agilent Technologies, USA) during the in vitro transcription process. 1.65 μg of Cy3-labeled cRNA was fragmented at 60° C. for 30 minutes. Correspondingly fragmented labeled cRNA was then pooled and hybridized to Agilent Mouse GE 4×44K V2 Microarray (Agilent Technologies, USA) at 65° C. for 17 h. After washing, the microarrays were scanned with an Agilent microarray scanner (AgilentTechnologies, USA) at 535 nm for Cy3. The array image was analyzed by the Feature Extraction software version 10.7.1.1 using default settings. The microarray following ErbB4 knockdown were deposited at GEO, under accession number GSE69897.

### Results

(i) Co-Culture with Endothelial Cells Induces Aspects of Maturation in Mouse Embryonic Stem Cell-Derived Cardiomyocytes

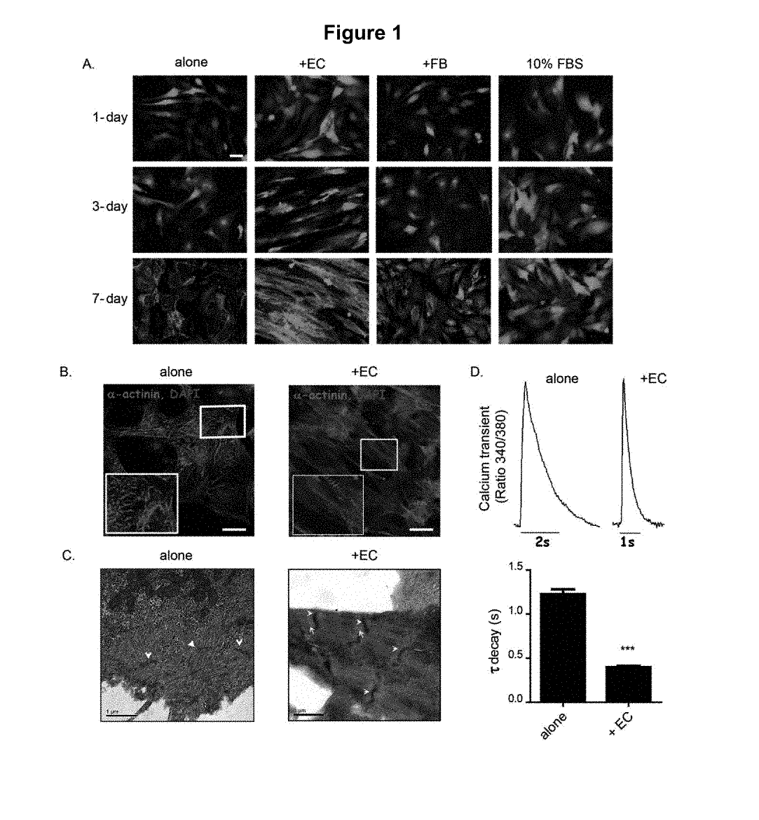

Mouse embryonic stem (mES) cells, stably transfected with a cardiac-specific alpha-MHC-driven EGFP reporter (Takahashi et al., Circulation, 2003, 107, 1912-1916), were differentiated into cardiomyocytes (CMs) using the hanging drop method, yielding ˜15% mES-CMs (FIG. 5, Panel A to Panel B). These mES-CMs were purified by FACS and co-cultured with rat endothelial cells (ECs) (FIG. 5, Panel C) or mouse cardiac fibroblasts which had been previously seeded on the plate. It was observed that mES-CMs co-cultured with ECs took on an elongated cell shape and improved their alignment, compared to when they were cultured alone (FIG. 1, Panel A).

During development, rodent heart maturation is characterized by a switch of fetal-specific myosin heavy chain β-isoform (β-MHC) to the α-isoform (α-MHC) that is predominantly expressed in adult rodent hearts (Lompré et al., J. Biol. Chem., 1984, 259, 6347-6446). ES-CMs were sorted out from co-culture by FACS, based on EGFP⁺ signal, and the α-/β-MHC ratio was assessed by real time RT-PCR. A higher α-/β-MHC ratio was found in ES-CMs which had been co-cultured with ECs, compared to ES-CMs cultured alone (FIG. 5, Panel E), reflecting a change of ES-CM maturity following co-culture.

Both immunofluorescence and ultrastructural analyses of co-cultured mES-CMs revealed myofibrils with properly aligned Z-bands, whereas ES-CMs cultured alone contained only nascent myofibrils and poorly aligned, immature Z-bodies (FIG. 1, Panel B to Panel C). I-bands, a marker of the mature phenotype, could only be visualized in the EC co-culture group (FIG. 1, Panel C). Calcium oscillations may also be used to characterize the maturity of pluripotent cell-derived cardiomyocytes. Therefore, calcium oscillations of co-cultured ES-CMs were measured using Fura-2, a Ca²⁺-sensitive dye. The rising slope and time to peak were not significantly different between the two groups, however a faster τ decay indicates improved kinetics of Ca²⁺ clearance from the cytosol of ES-CMs cultured with ECs (FIG. 1, Panel D). These data are supported by the elevation of the sarcoplasmic reticulum Ca²⁺ ATPase, Serca2a in co-cultured ES-CMs (FIG. 5, Panel F).
(ii) Delivery of Four microRNAs Induces Several Maturation-Associated Changes in Mouse ES-CMs

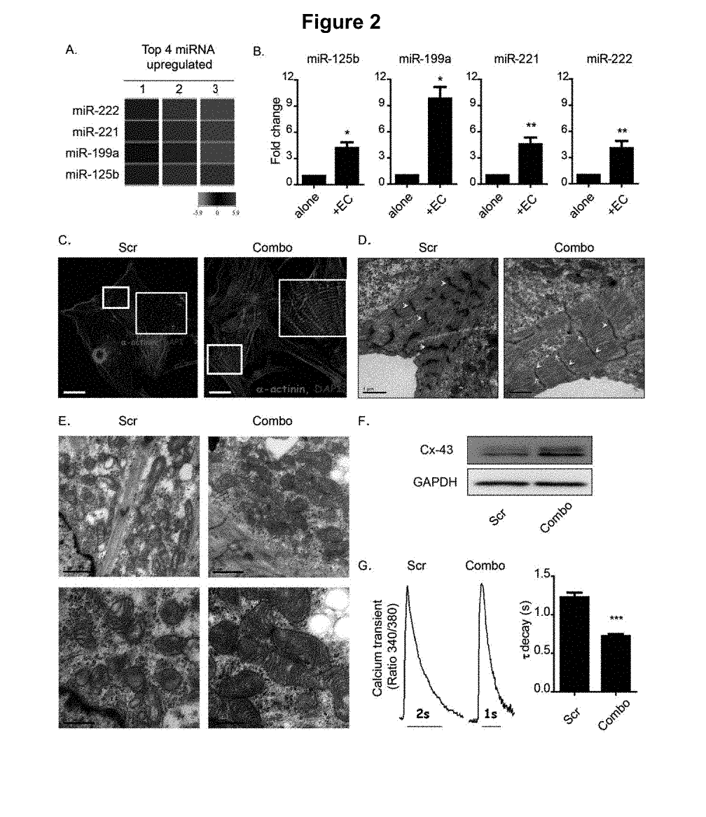

In order to compare miRNA expression profiles between EC co-cultured ES-CMs and those cultured alone, FACS was used to isolate ES-CMs from co-culture before extracting RNA and performing an miRNA microarray (FIG. 6, Panel A). Candidates that were most differentially upregulated in EC co-cultured ES-CMs in three independent experiments were chosen: miR-125b-5p, miR-199a-5p, miR-221, and miR-222 (FIG. 2, Panel A). The expression levels of these miRNAs were then confirmed using quantitative RT-PCR (FIG. 2, Panel B and FIG. 6, Panel B). Interestingly, these miRNAs also showed elevation during the embryonic stage of murine heart development (FIG. 6, Panel C). All of these miRNAs have been previously linked to cardiovascular disease in different roles (Cardinali et al., PLoS One, 2009, 4, e7607; Ge et al., J. Cell Biol., 2011, 192, 69-81; Small et al., Circulation, 2010, 121, 1022-1032; van Rooij et al., Proc. Natl. Acad. Sci. USA, 2006, 103, 18255-18260; Wong et al., PLoS One, 2012, 7, e36121).

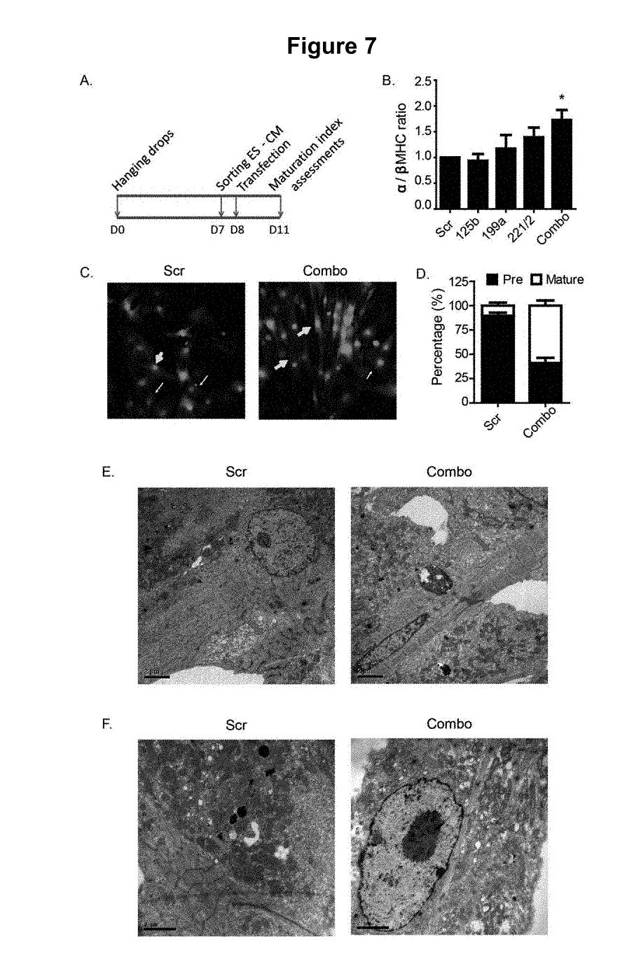

Each of these miRNAs were next tested in gain-of-function assays using synthetic miRNA precursors (FIG. 7, Panel A), using the β-to-α-MHC switch as a marker of maturation. Transfecting individual miRNAs had no significant effect on the α-/β-MHC ratio (FIG. 7, Panel B). However, since it has been reported that the combined overexpression of miRNAs is necessary to exert their particular function (Hu et al., Circulation, 2011, 124, S27-S34; Miyoshi et al., Cell Stem Cell, 2011, 8, 633-638), it was reasoned that the addition of these four miRNAs together (miR-combo) may be necessary to exert their effects. As suspected, a significant enhancement of the α-/β-MHC ratio was seen in ES-CMs transfected with miR-combo (FIG. 7, Panel B), reflecting a more mature phenotype. On the basis of these findings, these four miRNAs in combination for further experiments were used.

Examined next was whether transfection with the miR-combo could replicate the changes in mES-CM morphology and calcium handling observed during co-culture with endothelial cells. Indeed, miR-combo transfected mES-CMs showed a more elongated structure than scramble transfected controls (FIG. 7, Panel C) and contained more aligned, mature myofibrils in the cytoplasm (FIG. 2, Panel C and FIG. 7, Panel D), similar to those observed during EC co-culture. miR-combo transfected mES-CMs also showed more organized sarcomeric structures with the presence of I bands (FIG. 2, Panel D and FIG. 7, Panel E) and mitochondrial inner membranes with more well-formed cristae (FIG. 2, Panel E and FIG. 7, Panel F). In addition, an increased protein level of the cardiomyocyte gap junction component CONNEXIN-43 was also measured (FIG. 2, Panel F). Examination of calcium transients revealed a significant increase in the decay rate of Ca²⁺ clearance, as evidenced by a decrease in τ decay time (FIG. 2, Panel G). All of these findings point to an improvement in the maturity status of mES-CM following miR-combo delivery.
(iii) miR-Combo Promotes Several Maturation-Associated Changes in Human ES-CMs

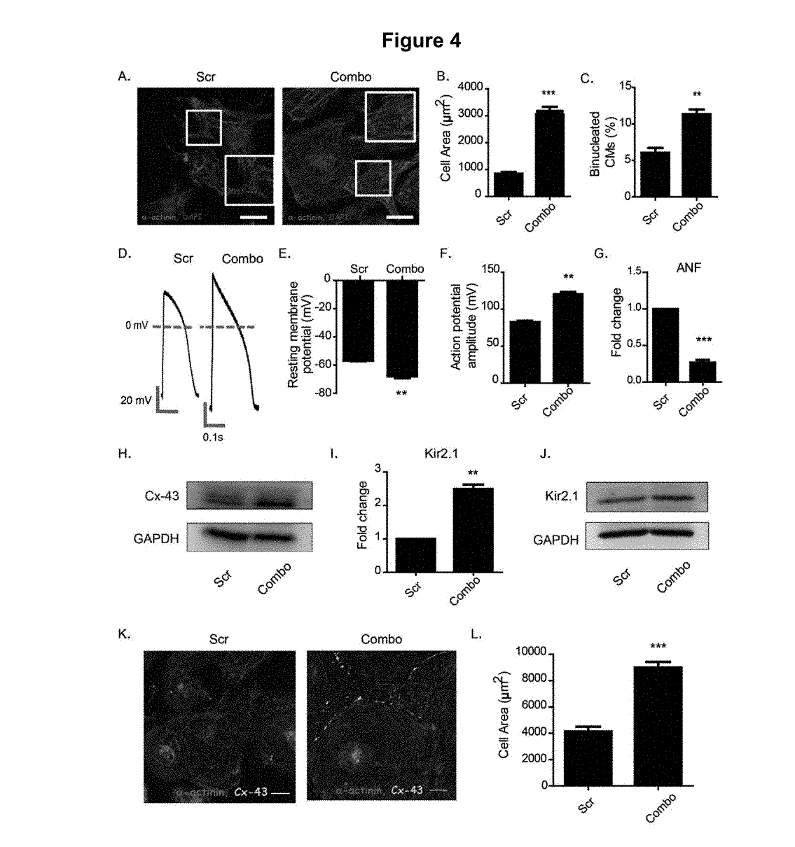

Human adult cardiomyocytes are an essential tool for scientific research, disease modeling and drug discovery. Yet, obtaining primary cells for research is exceedingly difficult. Since miR-125b-5p, miR-199a-5p, miR-221, and miR-222 are all evolutionarily conserved between mice and humans, the aim was to examine whether the findings from murine ES cells could also apply to the human system. Therefore, cardiac differentiation of H9 ES cells (Yang et al., J. Mol. Cell Cardiol., 2014, 72, 296-304) was induced and an extensive characterization of hES-CM maturity was carried out following miR-combo delivery. In a similar manner to mES-CMs, hES-CMs showed several morphological changes associated with increased maturation. hES-CMs transfected with the miR-combo showed more organized sarcomeres, a larger cell area, and a higher binucleation ratio compared to scramble miRNA transfected controls, although they still retained a rounded cell shape (FIG. 4, Panel A to Panel C). These changes were also accompanied by a more negative resting membrane potential and a larger amplitude of action potential, as measured by whole cell patch clamp (FIG. 4, Panel D to Panel F), although still falling short of the electrophysiological performance expected from adult human cardiomyocytes. In addition, the expression level of several genes and proteins, which are known to be differently expressed in fetal and adult CMs, were examined. Data showed lower expression of ANF and higher expression of CONNEXIN-43 and KIR2.1 following miR-combo delivery (FIG. 4, Panel G to Panel J). All of these factors indicate that hES-CM take on a more mature phenotype following miR-combo delivery.

In order to form a useful platform for future research, ES-CMs would ideally show a long-term improvement in maturation, rather than a transient effect. Therefore, it was sought to investigate the effects of miR-combo delivery after a longer culture period. hES-CMs were followed for two months after one single transfection with miR-combo and it was found that, after two months, a more organized distribution of Connexin-43 and enlarged cell size was still clearly visible in miR-combo treated hES-CMs, compared to controls which were cultured for the same period of time. (FIG. 4, Panel K and Panel L).

### Example 2

### Suppression of ErbB4 Induced Aspects of Maturation in Embryonic Stem Cell-Derived Cardiomyocytes

miRNAs are known to post-transcriptionally repress the expression of target genes. Therefore, target prediction analysis of miR-125b-5p, miR-199a-5p, miR-221, and miR-222 was carried out using TargetScan. TargetScan revealed that the 3′UTR of ErbB4 is a common target of these four miRNAs (FIG. 8, Panel A). Indeed, it was confirmed that there is a significant reduction in the expression of ErbB4 in ES-CMs following EC co-culture and following miR-combo delivery (FIG. 3, Panel A to Panel C). ErbB4 expression was also found to decline during the prenatal stage of cardiac development, inversely correlated with the expression of these four miRNAs (FIG. 3, Panel D).

Luciferase assays were performed to demonstrate that all four miRNAs discussed herein target ErbB4, and siRNA knockdown of ErbB4 partially recapitulated the effects of miR-combo.

In order to verify that the four miRNAs noted above can indeed target ErbB4, the ErbB4 3′UTR sequence was cloned and ligated behind the luciferase gene. It was found that each individual miRNA can significantly reduce luciferase activity, suggesting that each miRNA in the miR-combo can target ErbB4 (FIG. 3, Panel E).

Since miR-combo treatment appears to target ErbB4 expression, it was sought to examine whether the same maturation-associated changes could be observed following knockdown of ErbB4. Therefore, ES-CMs were transfected with two different ErbB4 siRNAs (FIG. 8, Panel B) and the same assays were used to assess the maturity indices of each group. A higher α-/β-MHC ratio (FIG. 3, Panel F), increased gene and protein expression of CONNEXIN-43 (FIG. 3, Panel C to Panel H) and more aligned sarcomeres following ErbB4 knockdown (siErbB4, FIG. 3, Panel I) were found. Notably, the degree of ES-CM maturity correlated with the knockdown efficiency of ErbB4 by the two different siRNAs, with the more efficient siRNA achieving the more mature characteristics. Overall, these data indicate that knockdown of ErbB4, as a target of miR-combo, alters many aspects of ES-CM morphology, gene expression and protein expression, in accordance with a more mature phenotype.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A method for enhancing cardiomyocyte maturation, the method comprising: (i) transfecting into immature cardiomyocytes one or more microRNA oligonucleotides, which are selected from the group consisting of a miR-125b-5p oligonucleotide, a miR-199a-5p oligonucleotide, a miR-221 oligonucleotide, and a miR-222 oligonucleotide; thereby producing mature cardiomyocytes.

2. The method of claim 1, wherein the method comprises transfecting into the immature cardiomyocytes a combination of the miR-125b-5p oligonucleotide, the miR-199a-5p oligonucleotide, the miR-221 oligonucleotide, and the miR-222 oligonucleotide.

3. The method of claim 1, wherein the immature cardiomyocytes are derived from embryonic stem cells.

4. The method of claim 3, wherein the embryonic stem cells are human embryonic stem cells.

5. The method of claim 3, wherein the embryonic stem cells are mouse embryonic stem cells.

6. The method of claim 1, wherein the immature cardiomyocytes are co-cultured with endothelial cells.

7. The method of claim 6, wherein the immature cardiomyocytes and the endothelial cells are of the same species.

8. The method claim 1, wherein the method further comprising: (ii) measuring the level of major histocompatibility complex (MHC) alpha chain and/or the level of MHC beta chain in the cardiomyocytes after step (i); and (iii) determining the maturation level of the cardiomyocytes based on the level of the MHC alpha chain and/or the level of the MHC beta chain.

9. The method of claim 8, wherein the maturation level of the cardiomyocytes is determined based on the ratio between the level of the MHC alpha chain and the level of the MHC beta chain.

10. The method of claim 1, where the method further comprising transplanting the mature cardiomyocytes produced in step (i) to a subject in need thereof.

11. The method of claim 1, wherein the method further comprising contacting the mature cardiomyocytes produced in step (i) with a candidate agent to determine the impact of the candidate agent on the mature cardiomyocytes.

12. A method for enhancing cardiomyocyte maturation, the method comprising: (i) suppressing ErbB4 in immature cardiomyocytes, thereby producing mature cardiomyocytes.

13. The method of claim 12, wherein the immature cardiomyocytes are derived from embryonic stem cells.

14. The method of claim 13, wherein the embryonic stem cells are human embryonic stem cells or mouse embryonic stem cells.

15. The method of claim 12, wherein the immature cardiomyocytes are co-cultured with endothelial cells.

16. The method of claim 15, wherein the immature cardiomyocytes and the endothelial cells are of the same species.

17. The method of claim 12, wherein step (i) is performed by transfecting into the immature cardiomyocytes an interfering RNA that targets ErbB4.

18. The method of claim 12, wherein the method further comprising: (ii) measuring the level of major histocompatibility complex (MHC) alpha chain and/or the level of MHC beta chain in the cardiomyocytes after step (i); and (iii) determining the maturation level of the cardiomyocytes based on the level of the MHC alpha chain and/or the level of MHC beta chain.

19. The method of claim 12, where the method further comprising transplanting the mature cardiomyocytes produced in step (i) to a subject in need thereof.

20. The method of claim 12, wherein the method further comprising contacting the mature cardiomyocytes produced in step (i) with a candidate agent to determine the impact of the candidate agent on the mature cardiomyocytes.
